(19) United States
(12) Patent Application Publication
(10) Pub. No.: US 2013/0177543 A1
(43) Pub. Date: Jul. 11, 2013

(54) Natural Means of Augmenting Endogenous Stem Cell Numbers

(71) Applicant: Creative Medical Health Inc., Phoenix, AZ (US)

(72) Inventors: Amit Patel, Salt Lake City, UT (US); Annette Marleau, San Diego, CA (US)

(73) Assignee: CREATIVE MEDICAL HEALTH INC., Phoenix, AZ (US)

(21) Appl. No.: 13/717,691

(22) Filed: Dec. 17, 2012

Related U.S. Application Data
(60) Provisional application No. 61/578,089, filed on Dec. 20, 2011.

(51) Int. Cl. A61K 31/593; A61K 45/06; A61K 31/737; A61K 36/38; A61K 36/81; A61K 36/28

(52) U.S. Cl. CPC A61K 31/593; A61K 36/81; A61K 36/28; A61K 31/737; A61K 36/38; A61K 45/06
USPC 424/94.1; 424/725; 424/764; 514/167; 424/195.17; 514/690; 424/729

(57) ABSTRACT

Disclosed are compositions of matter, uses, and therapeutic interventions directed towards modulation of stem cell activity in a mammal. In particular the invention provides compositions whose end result modulates health, wellbeing, longevity and function of organ systems. Within the scope of the invention are biologically active extracts, components thereof, and compositions (such as cosmetic or pharmaceutical preparations) made comprising such.

### CROSS-REFERENCE TO RELATED APPLICATIONS

This application claims priority to co-pending U.S. Provisional Application Ser. No. 61/578,089, filed Dec. 20, 2011, which is expressly incorporated herein by reference in its entirety.

### FIELD OF THE INVENTION

The invention pertains to the field of stem cell modulation. Specifically, the invention relates to the use of natural compounds for modulation of stem cell function. More specifically, the invention relates to the use of combinations of a) Ashwagandha; b) Milk Thistle; c) Vitamin D3; d) Fucoidan; and e) Garcinia Indica.

### BACKGROUND

Several agents capable of modulating stem cell mobilization have been described in the art including G-CSF [1], beta glucan [2-5], parathyroid hormone, CXCR4 receptor antagonists [6], and complement components [7]. It is well known in the field that stem cell mobilization is a physiological response to injury, and that augmented numbers of stem cells are associated with increased recovery in certain conditions. Therefore the rationale exists that artificially augmenting stem cell mobilization should have therapeutic effects on injury. This has been demonstrated in stroke, in which Boy et al [8]. studied three cohorts of patients (8, 6, and 6 patients per cohort) who have been treated with 2.5, 5, or 10 μg/kg body weight of G-CSF for 5 consecutive days within 12 hrs of onset of acute stroke in addition to standard thrombolytic therapy. Mobilization of CD34(+) cells was observed with no concomitant changes in blood chemistry, except for an increase in the leukocyte count up to 75,500/μl. Neuroradiological and neuropsychological follow-up studies did not disclose any specific G-CSF toxicity. VGM findings indicated substantial atrophy of related hemispheres, a substantial increase in the CSF space, and a localized increase in parenchyma within the ischemic area in 2 patients. Additionally, improved behavioral symptoms were noted.

Other manipulators of stem cells numbers are well known in the art, the first manipulation of stem cells commercially by pharmaceutical means was the development of, and introduction into clinical practice of erythropoietin (EPO), a kidney-produced hormone that stimulates red blood cell production from bone marrow hematopoietic stem cells. Originally named in 1948 and cloned in 1985 [9], erythropoietin has developed into a multibillion dollar drug. For example, in 2004, Amgen's version of this hormone, called Epogen had US sales of $2.6 billion, Roche's brand of EPO had European sales of $1.7 billion, and Johnson and Johnson's had sales of $3.6 billion [10]. EPO is typically used in treatment of patients with chronic renal failure, drug induced anemias, and to reduce number of transfusions needed in surgical patients [11]. Interestingly, in addition to its ability to stimulate erythrocyte production, scientists started to identify non-hematopoietic activities of this hormone. Since EPO has been clinically used for decades, transition of EPO into non-hematopoietic regenerative uses was relatively simple since it did not require extensive safety testing. For example, the erythropoietin receptor has been described on endothelial cells [12], macrophages [13], neurons [14, 15], glial cells [16], and cardiomyocytes [17]. What role could it play on these cells? Well animal models of demonstrated that EPO administration is beneficial in conditions ranging from injury induced erectile dysfunction [18], to stroke [18], to heart failure [19-22]. Clinical trials have also been conducted using EPO for non-hematopoietic conditions.

One particular patent of interest is U.S. Pat. No. # 6,165,783 entitled “Erythropoietic mediated neurogenesis.” This patent has the earliest priority date of Oct. 24, 1997, and was issued Dec. 26, 2000. Dr. Samuel Weiss, Professor and Director of the Hotchkiss Brain Institute http://www.hbi.ucalgary.ca from Calgary Canada is the first inventor. In its claims the patent covers primarily: 1) Inducing differentiation of neural stem cells into neurons by stimulating proliferation of the neural stem cells with a proliferation inducing agent and adding EPO so as to allow for differentiation. 2) A treatment method for neurodegenerative diseases or injury to the brain by coaxing multipotent neural stem cells to differentiate into neurons through the addition of EPO. 3) Treatment of brain injury/degeneration through addition of cells that have been stimulated with EPO and/or other factors such as EGF. Dr. Weiss is currently collaborating with the Calgary based company Stem Cell Therapeutics (www.stemcellthera.com) in clinically translating the use of EPO and hCG for treatment of stroke. Unfortunately, due to the international clinical moratorium on EPO trials for neurological conditions, this work was stopped.

Attempts have been made to generate EPC analogues that lack possibility of hemorrhage. U.S. Pat. No. # 6,531,121 entitled “Protection and enhancement of erythropoietin-responsive cells, tissues and organs” of which Dr Michael Brines, CSO of Warren Pharmaceuticals (http://www.warrenpharma.com) is primary inventor, teaches the use of EPO in protecting tissues. This may be a different biological mechanism than stimulation of stem cells but is nevertheless interesting. The patent essentially covers using a type of EPO that does not stimulate erythropoiesis but confers protection of viability and function to a variety of tissues including retinal, cardiac, and endothelial tissues. Warren Pharmaceuticals has taken a partnering strategy for commercialization and clinical implementation of its EPO candidates. According to the company website, Warren is currently partnered with H. Lundbeck A/S, The BankInvest Group, and Shire plc. H. Lundbeck A/S (www.lundbeck.com) entered into a license and research agreement with Warren in October of 2001. The collaboration was aimed primarily at treatment of CNS diseases. In October of 2007, Lundbeck initiated Phase I trials using a chemically modified (carbamoylated) form of EPO to treat patients suffering from stroke. The partnership with The BankInvest Group (www.bankinvest.com) started also in October 2001. According to the Warren website, the BankInvest Group purchased an equity position in Warren, although we could not find any specifics regarding the size of the investment. In September of 2006, the pharmaceutical giant Shire plc (www.shire.com) purchased rights for all other Warren tissue protective IP besides in the area of CNS.

U.S. Pat. No. # 7,129,267 entitled “Methods for SHP1 mediated neuroprotection” and assigned to Janssen Pharmaceutica N.V. accepts as a given that EPO is neuroprotective and/or neuroregenerative but goes on to cover methods of augmenting EPO activity in the nervous system. Specifically the patent has 1 claim, which covers “A method for increasing the potency of erythropoietin (EPO) in a subject in need thereof, comprising the step of administering to the subject an effective dose of a composition that decreases the tyrosine phosphatase activity of SHP1 in a cell of the nervous system of the subject, wherein said composition is selected from the group consisting of a compound of Formula (I); ##STR00004## a compound of Formula (II); ##STR00005## and a compound of Formula (III); ##STR00006##” Essentially, the patent is stating that the inhibition of the phosphatase SHP-1 is involved in the inhibition of EPO signaling in neurons, so if you inhibit the inhibitor you get augmentation of the effect. The patent has 4 compounds that inhibit SHP-1 and stimulate EPO triggered neuroregenerative activites. This is particularly of interest since astrocytes in the brain naturally can produce EPO [23], therefore it may be possible that these SHP-1 inhibiting compounds may be used as a monotherapy for regeneration. Granulocyte colony stimulating factor (G-CSF) is a multibillion dollar a year drug currently used for the treatment of neutropenia in patients after bone marrow transplantation or chemotherapy, as well as mobilization of hematopoietic stem cells into the periphery. Initially, G-CSF was studied because of its known ability to induce the selective differentiation of granulocytic colonies when bone marrow stem cells where plated in semisolid media. Eventually molecular cloning of the gene encoding this cytokine was accomplished, thus allowing for its mass production. G-CSF expressed in E. coli is sold by Amgen under the trade name Neupogen®, whereas a pegylated form of it, which improved serum half-life is sold under the name Neulasta®. Some of Amgen's key patents on G-CSF expired in March 2006. Accordingly there is need for identification of new ways to use this molecule that has already demonstrated so much clinical benefit, at least in the area of hematology.

In 2005 the 1-year results of the Front-Integrated Revascularization and Stem Cell Liberation in Evolving Acute Myocardial Infarction by Granulocyte Colony-Stimulating Factor (FIRSTLINE-AMI) Trial were reported [24]. In this study 30 patients with ST-elevation myocardial infarction who were successfully revascularized where divided so that half the patients received subcutaneous G-CSF at 10 μg/kg body weight for 6 days in addition to standard care, whereas the other 15 received standard care only. It was demonstrated that administration of the G-CSF, at least in this small group of patients, was safe. This is a major concern since the G-CSF increases CD34s 20-fold higher than normal, as well as causing a profound upregulation of neutrophils. Since neutrophils are inflammatory, one may have been worried about possible inflammatory exacerbation of the infarct. Luckily this was not the case. 4 months after the infarct, the group that received G-CSF possessed a thicker myocardial wall at the area of infarct, as compared to controls. This was sustained over a year. Statistically significant improvements in ejection fraction, as well as inhibition of pathological remodeling was observed in comparison to controls. You may see the figure to the right which demonstrates the statistically significant improvement in ejection fraction. This study strongly encouraged the future investigation of this modality. Interesting, a subsequent study involving 114 patients, 56 treated and 58 control demonstrated “no influence on infarct size, left ventricular function, or coronary restenosis” [25]. There may be a variety of reasons to explain the discrepancy between the trials. One most obvious one is that the mobilization was conducted immediately after the heart attack, whereas it may be more beneficial to time the mobilization with the timing of the chemotactic gradient released by the injured myocardium. This has been used to explain discrepancies between similar regenerative medicine trials [26]. Supporting this possibility is a study in which altered dosing was used for the successful improvement in angina [27]. Furthermore, a recent study last year demonstrated that in 41 patients with large anterior wall AMI an improvement in LVEF and diminished pathological remodeling was observed [28]. Thus there are numerous studies demonstrating “clinical signals”, however it appears that some optimization of patient criteria is needed before mobilization may become an effective therapy. Along these lines, there is an interesting hypothesis that in some patients, especially those at risk of AMI, the stem cell compartment is “exhausted”. For example, in patients with cardiac risk factors, CD34 stem cells appear to have shorter telomeres [29-31].

In addition to cardiac conditions, G-CSF mobilization has also been used for the treatment of stroke. Theoretically many of the same concepts discussed also apply to infarcts of the brain, or stroke. If anything, the concepts are even stronger in stroke since clinical studies have demonstrated a significant correlation between endogenous mobilization and recovery [32, 33]. In a clinical study of 7 treated and 3 placebo stroke patients, improvement in neurologic functioning between baseline and 12-month follow-up was statistically significantly greater in the G-CSF group than in the control group (NIHSS: 59% change in the mean G-CSF group score v. 36% in the mean control group score, ESS: 33% v. 20%, EMS: 106% v. 58%, BI: 120% v. 60%). Additionally, patients reported no serious adverse effects as a result of the G-CSF treatment [34]. Preclinical animal models support the use of G-CSF not only for its ability to mobilize stem cells but also for direct neuroprotective activities [35, 36].

As one can imagine, several of the non-hematopoietic uses of G-CSF patents are held by Amgen. For example, U.S. Pat. No. 7,220,407 covers the use of G-CSF administration after a person has had a heart attack and is successfully revascularized. The patent covers the use of G-CSF to: A) Prevent post AMI reduction in ventricular wall thickness; B) Reduce myocardial scarring; C) Reduce necrosis and D) Improve patient outcome. The patent claims G-CSF after revascularization, G-CSF after bypass surgery, combinations of G-CSF with other cytokines, as well as specific concentrations of G-CSF. Supporting examples for the patent include a porcine 90 minute proximal circumflex artery balloon occlusion model in which G-CSF was able to inhibit myocardial scarring, pathological remodeling and maintain LVEF.

Another pharmaceutical that has been used in the field of increasing numbers of circulating stem cells is growth hormone (GH), a 191-amino acid, single chain polypeptide hormone that is naturally produced by the anterior pituitary. As the name implies, it is involved in the growth of children. Several biological activities include instructing the division of chondrocytes at the epiphyses of long bones, stimulation of IGF-1, and stimulation of organ growth. Deficiencies of GH are associated with short stature in children, in adults, deficiency causes lose of muscle, gain of adipose tissue around waistline, and increased cardiovascular risk. Recombinant GH, has been available since 1985 made in various forms by Genentech (Protropin and Nutropin) and Lilly (Humatrope) and approved in the USA for: Pediatric Growth Hormone Deficiency, Idiopathic Short Stature, Adult Growth Hormone Deficiency, Turner Syndrome, and Chronic Renal Insufficiency.

A subset of “unproven” or “complementary” medicine use GH administration for “anti-aging” activities [37]. Numerous studies with GH have been performed that indirectly or directly support ability of this hormone to induce regenerative effects possible via stem cell activation. The commercial implications of non-growth uses of GH have caught the attention of several companies. For example, Applied Research Systems ARS Holding N.V. which is owned by Serona, is the named assignee on U.S. Pat. No. # 6,348,444, entitled “Human growth hormone to stimulate hematopoiesis and immune reconstitution after hematopoietic stem cell transplantation in humans.” This patent covers the use of GH and various variants of it for the stimulation of human hematopoiesis either after transplantation or in a myeloablated state. This patent has been clinically validated by a randomized, double-blind, placebo-controlled study of patients who were hematopoietically compromised after intensive chemotherapy [38 ]. Patients treated with GH had a significantly faster recovery of platelets to 25×10(9)/1 (median of 16 versus 19 days; P=0.03) compared to the placebo-controlled arm. Time to relapse did not differ significantly between arms. This is important since one argument could be that the GH would accelerate relapse of neoplasia. Additionally, no abnormal growth or acromegaly was observed after treatment.

As seen in the above experiences, numerous agents that stimulate increased numbers of stem cells in circulation have applicability in “naturally regenerating” injured tissues. Unfortunately, the use of such pharmacological/pharmaceutical approaches is limited by toxicities, unintended adverse effects, and economical perspectives. Additionally, these agents all induce an acute rise in stem cell numbers, which does not replicate physiological healing processes. It is the scope of the present invention to provide nutraceutical means of controlling damage to the stem cell compartment by providing natural based support, as well as augmenting numbers of stem cells in circulation.

### SUMMARY OF THE INVENTION

The teachings herein are directed to methods of augmenting stem cell numbers in circulation comprising administering to a mammal a composition comprising of one or more ingredients selected from the group consisting of: a) Ashwagandha; b) Milk Thistle; c) Vitamin D3; d) Fucoidan; and e) Garcinia Indica.

### DETAILED DESCRIPTION

Embodiments of the present invention are described below. It is, however, expressly noted that the present invention is not limited to these embodiments, but rather the intention is that modifications that are apparent to the person skilled in the art and equivalents thereof are also included.

The invention teaches various compositions of herbs, vitamins and natural products for stimulation of stem cell numbers in circulation. Therapeutic effects of augmenting circulating stem cell numbers derived from data demonstrating that augmentation of said numbers results in a beneficial effect in degenerative conditions such as stroke, ischemic cardiomyopathy, critical limb ischemia, and myocardial infarction. In the area of tissue regeneration, one application that comes to mind is mobilization of bone marrow stem cells in order to increase the amount of cells that enter the area of tissue injury. For example, it is known in many situations that injured tissue releases chemotactic signals that induce “homing” of stem cells. On example of this is after myocardial infarction there is a documented rise in plasma VEGF which is associated with stem cell mobilization and entry into the peripheral circulation, theoretically on the way to the damaged myocardium [39-42]. Such injury induced stem cell mobilization has also been documented in stroke [32, 33], liver failure [43], renal ischemia reperfusion injury [44], and transplantation [45]. Now the question is, if tissue injury can promote accelerated mobilization of stem cells to the injured area, and if in some cases more mobilization equals more protection from pathology [32, 33], then would it not make sense to simply use G-CSF administration to mobilize bone marrow stem cells with the idea of augmenting existing natural processes? Unfortunately numerous toxicities and expense limit this approach

In one particular embodiment, the therapeutic composition provided is a mixture of Ashwagandha, Milk Thistle, Vitamin D3, Fucoidan and Garcinia Indica. Said ingredients can be mixed at a ratio determined by one skilled in the art to elicit the desired amount of stem cell mobilization. Mobilization of stem cells may be assessed by techniques known in the art, said techniques include flow cytometry to assess number of cells with a size, density and/or phenotypic profile of the desired stem cell population. It is apparent to one of skill in the art that if augmented numbers of hematopoietic stem cells are desired, then the practitioner may assess levels of CD34, CXCR4, CD133, stem cell antigen or aldehyde dehydrogenase bright cells. Additionally, methods are known in the art to assess circulating stem cell numbers based on ability of said cells to generate differentiated progeny in vitro. For example, if the practitioner of the invention is seeking to identify endothelial progenitor cells (EPC), numbers of these cells in circulation may be assessed by flow cytometry to identify markers such as VEGFR2, or alternatively, to identify ability of the cells to form endothelial colonies in vitro. Said endothelial colonies may be quantified by morphology or ability to uptake acetylated LDL.

For the purposes of the current invention, within the definition of the term “stem cell” we include not only hematopoietic, endothelial, and mesenchymal stem cells, but also progenitor cells that have been committed to a certain lineage, for example, myeloid progenitor cells or lymphoid progenitor cells. Other tissue committed cells in circulation are also covered by our definition of the word “stem cells”.

In one specific embodiment, the therapeutic composition contains approximately 5-3000 mg ashwagandha, 50-10000 IU vitamin D3, 10-10000 mg fucoidan, and 5-5000 mg of garcinia indica. In one specific embodiment, the therapeutic composition contains approximately 50-300 mg ashwagandha, 500-2000 IU vitamin D3, 100-1000 mg fucoidan, and 50-500 mg of garcinia indica. In another embodiment said therapeutic composition is comprised of approximately 100 mg ashwagandha, 1000 IU vitamin D3, 500 mg fucoidan, and 250 mg of garcinia indica. In certain embodiments ingredients may be added or extracted. For example, in patients with known high levels of oxidative stress, such as dialysis patients, antioxidants may be added to the therapeutic composition. Said antioxidants may be natural, synthetic, or recombinant. Specific antioxidants are well known in the art and may be determined by experimentation. Example antioxidants include flavanoids, genistein, 1-carnosine; carotenoid; co-enzyme Q10, green tea or extract thereof, vitamin A, vitamin B, vitamin C, vitamin E, zinc, selenium, resveratrol, lipoic acid, chlorophyll, gogji berries and extracts thereof, fish oil, and a superoxide dismutase.

The compositions of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the formulation and not deleterious to the user. The embodiments of the compositions described in the invention can be formulated in any suitable product form. Such product forms include, but are not limited to liquid, aerosol spray, cream, dispersion, emulsion, foam, gel, lotion, ointment, powder, solid and solution. The present compositions preferably include a vehicle. A useful vehicle is one that is medically acceptable for ingestion or topical application. Useful vehicles may include, but are not limited to, one or more aqueous systems, glycerin, alcohols, fatty alcohols, fatty ethers, fatty esters, polyols, glycols, vegetable oils, mineral oils, water, fruit juices, cultured dairy products, (e.g., yogurt), dairy products (e.g., milk), carbonated or non-carbonated beverages, and mixtures of the aforesaid. The powder compositions of the invention may be conveniently incorporated into a variety of liquids or solid compositions including, but not limited to, powders and other solids, liquid beverages (teas, mineral water, milk and other dairy products), capsules, tablets, ointments, skin creams, personal care products, pastas, soups, nutrient bars, cookies, breads, and other bakery items

For use in wound healing or treatment of other acute or chronic conditions of the epidermis, mixture of the ingredients Ashwagandha, Milk Thistle, Vitamin D3, Fucoidan and Garcinia Indica is formulated for topical administration. The vehicle for topical application may be in one of various forms, e.g. a lotion, cream, gel, ointment, stick, spray, or paste. These product forms can be formulated according to well known methods. They may comprise various types of carriers, including, but not limited to, solutions, aerosols, emulsions, gels, and liposomes. The carrier may be formulated, for example, as an emulsion, having an oil-in-water or water-in-oil base. Suitable hydrophobic (oily) components employed in emulsions include, for example, vegetable oils, animal fats and oils, synthetic hydrocarbons, and esters and alcohols thereof, including polyesters, as well as organopolysiloxane oils. Such emulsions also include an emulsifier and/or surfactant, e.g. a nonionic surfactant, such as are well known in the art, to disperse and suspend the discontinuous phase within the continuous phase. The topical formulation may contain one or more components selected from a structuring agent, a thickener or gelling agent, and an emollient or lubricant. Frequently employed structuring agents include long chain alcohols, such as stearyl alcohol, and glyceryl ethers or esters and oligo(ethylene oxide) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations may further include other components as known in the art, e.g. astringents, fragrances, pigments, skin penetration enhancing agents, sunscreens, etc.

In other embodiments, the nutraceutical compositions comprising of Ashwagandha, Milk Thistle, Vitamin D3, Fucoidan and Garcinia Indica may also be formulated for administration parenterally, transdermally, or by inhalation. An injectable composition for parenteral administration typically contains the active compound in a suitable IV solution, such as sterile physiological saline. The composition may also formulated as a suspension in a lipid or phospholipid, in a liposomal suspension, or in an aqueous emulsion.

The therapeutic properties of the various components of the composition have been previously described, however, utilization of these compositions for stimulation of stem cell function has not been reported. In the current invention, therapeutic compositions seem to be associated with additive/synergistic effects of the named ingredients to augment stem cell mobilization, as well as to stimulate function and inhibit stem cell inhibitory activities such as oxidative stress or inflammatory mediator production.

Ashwagandha administered at 500 mg three times per day has been reported by Sriranjini et al to normalize, or partially normalize balance in patients with cerebellar ataxia. (Sriranjini et al. Improvement of balance in progressive degenerative cerebellar ataxias after Ayurvedic therapy: a preliminary report. Neurol India. 2009 March-April; 57(2):166-71). Therapeutic effects at controlling viral hepatitis have been also reported for ashwagandha. When the herb was used in combination with two other natural ingredients in the proprietary composition livwin, a reduction of viral load was observed in patients with hepatitis B and C. Interestingly, the study was performed in double blind, placebo controlled setting (Keche et al. Efficacy and safety of livwin (polyherbal formulation) in patients with acute viral hepatitis: A randomized double-blind placebo-controlled clinical trial. Int J Ayurveda Res. 2010 October; 1(4):216-9). Improvement in endurance in athletes was also reported by administration of ashwagandha. In a clinical study, Sandhu et al (Effects of Withania somnifera (Ashwagandha) and Terminalia arjuna (Arjuna) on physical performance and cardiorespiratory endurance in healthy young adults. Int J Ayurveda Res. 2010 July; 1(3):144-9) demonstrated superior augmentation of exercise time as compared to placebo controls after administration of 500 mg of the herb twice a day.

Stimulation of immune modulatory activity was previously ascribed to the ashwagandha herb, however no clinical utilization in the context of therapeutics was performed. The immune modulatory activity was associated with Type 1 immunity, which is currently believed to inhibit stem cell activity. Thus the use of ashwagandha in the context of stem cell stimulation is counterintuitive in the context of the existing art. An indication that this herb may have potential to modulate the stem cell compartment was provided in a study by Davis and Kuttan (Immunopharmacol Immunotoxicol. 1999 November; 21(4):695-703. Effect of Withania somnifera on cytokine production in normal and cyclophosphamide treated mice). The investigators found that administration of an extract from the powdered root of the plant Withania somnifera (Family: Solanaceae) enhanced the levels of Interferon gamma (IFN-gamma) (75.87 pg/ml), Interleukin-2 (IL-2) (14.16 pg/ml).

In one embodiment of the invention, the nutraceutical composition is utilized as an adjunct to stem cell therapy. Numerous types of stem cell therapies exist and are known in the art that may be utilized in combination with the current invention. For example, said nutraceuticals can be used in combination with autologous bone marrow stem cell therapy, as currently practiced using devices such as the Harvest, Arteriocyte, Tissue Genesis, or Bio-met device.

The following references have been referred to in the text. The entire content of each reference is incorporated herein. Without limitation all of the cells, proteins, markers, assays, and methods can be used with the technology described herein and/or can be combined with the methods and materials described herein.

### REFERENCES

1. Patchen, M. L., et al., Mobilization of peripheral blood progenitor cells by Betafectin PGG-Glucan alone and in combination with granulocyte colony-stimulating factor. Stem cells, 1998. 16(3): p. 208-17.
2. Lin, H., et al., Maitake beta-glucan MD-fraction enhances bone marrow colony formation and reduces doxorubicin toxicity in vitro. International immunopharmacology, 2004. 4(1): p. 91-9.
3. Cramer, D. E., et al., Beta-glucan enhances complement-mediated hematopoietic recovery after bone marrow injury. Blood, 2006. 107(2): p. 835-40.
4. Cramer, D. E., et al., Mobilization of hematopoietic progenitor cells by yeast-derived beta-glucan requires activation of matrix metalloproteinase-9. Stem cells, 2008. 26(5): p. 1231-40.
5. Lee, H. M., et al., Impaired mobilization of hematopoietic stem/progenitor cells in C5-deficient mice supports the pivotal involvement of innate immunity in this process and reveals novel promobilization effects of granulocytes. Leukemia: official journal of the Leukemia Society of America, Leukemia Research Fund, U.K, 2009. 23(11): p. 2052-62.
6. Yannaki, E., et al., Hematopoietic Stem Cell Mobilization for Gene Therapy of Adult Patients With Severe beta-Thalassemia: Results of Clinical Trials Using G-CSF or plerixafor in Splenectomized and Nonsplenectomized Subjects. Molecular therapy: the journal of the American Society of Gene Therapy, 2011.
7. Ratajczak, M. Z., et al., The role of innate immunity in trafficking of hematopoietic stem cells-an emerging link between activation of complement cascade and chemotactic gradients of bioactive sphingolipids. Advances in experimental medicine and biology, 2012. 946: p. 37-54.
8. Boy, S., et al., Mobilisation of Hematopoietic CD34 Precursor Cells in Patients with Acute Stroke Is Safe—Results of an Open-Labeled Non Randomized Phase I/II Trial. PloS one, 2011. 6(8): p. e23099.
9. Jelkmann, W., Erythropoietin after a century of research: younger than ever. Eur J Haematol, 2007. 78(3): p. 183-205.
10. http://pubs.acs.org/cen/coverstory/83/8325/8325erythropoietin.html.
11. http://www.rxlist.com/cgi/generic/epoetin_ids.htm.
12. Anagnostou, A., et al., Erythropoietin receptor mRNA expression in human endothelial cells. Proc Natl Acad Sci U S A, 1994. 91(9): p. 3974-8.
13. Haroon, Z. A., et al., A novel role for erythropoietin during fibrin-induced wound-healing response. Am J Pathol, 2003. 163(3): p. 993-1000.
14. Konishi, Y., et al., Trophic effect of erythropoietin and other hematopoietic factors on central cholinergic neurons in vitro and in vivo. Brain Res, 1993. 609(1-2): p. 29-35.
15. Wang, L., et al., Treatment of stroke with erythropoietin enhances neurogenesis and angiogenesis and improves neurological function in rats. Stroke, 2004. 35(7): p. 1732-7.
16. Li, X., S. L. Gonias, and W. M. Campana, Schwann cells express erythropoietin receptor and represent a major target for Epo in peripheral nerve injury. Glia, 2005. 51(4): p. 254-65.
17. Wright, G. L., et al., Erythropoietin receptor expression in adult rat cardiomyocytes is associated with an acute cardioprotective effect for recombinant erythropoietin during ischemia-reperfusion injury. FASEB J, 2004. 18(9): p. 1031-3.
18. !!! INVALID CITATION !!!
19. Moon, C., et al., Erythropoietin reduces myocardial infarction and left ventricular functional decline after coronary artery ligation in rats. Proc Natl Acad Sci U S A, 2003. 100(20): p. 11612-7.
20. Ye, L., et al., Effects of rHu-EPO on myocyte apoptosis and cardiac function following acute myocardial infarction in rats. J Huazhong Univ Sci Technolog Med Sci, 2005. 25(1): p. 55-8.
21. Xu, B., et al., Recombinant human erythropoietin pretreatment attenuates myocardial infarct size: a possible mechanism involves heat shock Protein 70 and attenuation of nuclear factor-kappaB. Ann Clin Lab Sci, 2005. 35(2): p. 161-8.
22. van der Meer, P., et al., Erythropoietin induces neovascularization and improves cardiac function in rats with heart failure after myocardial infarction. J Am Coll Cardiol, 2005. 46(1): p. 125-33.
23. Masuda, S., et al., A novel site of erythropoietin production. Oxygen-dependent production in cultured rat astrocytes. J Biol Chem, 1994. 269(30): p. 19488-93.
24. Ince, H., et al., Prevention of left ventricular remodeling with granulocyte colony-stimulating factor after acute myocardial infarction: final 1-year results of the Front-Integrated Revascularization and Stem Cell Liberation in Evolving Acute Myocardial Infarction by Granulocyte Colony-Stimulating Factor (FIRSTLINE-AMI) Trial. Circulation, 2005. 112(9 Suppl): p. 173-80.
25. Zohlnhofer, D., et al., Stem cell mobilization by granulocyte colony-stimulating factor in patients with acute myocardial infarction: a randomized controlled trial. JAMA, 2006. 295(9): p. 1003-10.
26. Hill, J. M. and J. Bartunek, The end of granulocyte colony-stimulating factor in acute myocardial infarction? Reaping the benefits beyond cytokine mobilization. Circulation, 2006. 113(16): p. 1926-8.
27. Suzuki, K., et al., Effect of granulocyte colony-stimulating factor treatment at a low dose but for a long duration in patients with coronary heart disease. Circ J, 2006. 70(4): p. 430-7.
28. Leone, A. M., et al., Usefulness of granulocyte colony-stimulating factor in patients with a large anterior wall acute myocardial infarction to prevent left ventricular remodeling (the rigenera study). Am J Cardiol, 2007. 100(3): p. 397-403.
29. Brown, R. L., Stem cell exhaustion and atherosclerosis. J Anti Aging Med, 2003. 6(3): p. 279; discussion 280.
30. Thorin-Trescases, N., et al., Pathological aging of the vascular endothelium: are endothelial progenitor cells the sentinels of the cardiovascular system? Can J Cardiol, 2005. 21(12): p. 1019-24.
31. Sharpless, N. E. and R. A. DePinho, How stem cells age and why this makes us grow old. Nat Rev Mol Cell Biol, 2007. 8(9): p. 703-13.
32. Sobrino, T., et al., The increase of circulating endothelial progenitor cells after acute ischemic stroke is associated with good outcome. Stroke, 2007. 38(10): p. 2759-64.
33. Yip, H. K., et al., Level and value of circulating endothelial progenitor cells in patients after acute ischemic stroke. Stroke, 2008. 39(1): p. 69-74.
34. Shyu, W. C., et al., Granulocyte colony-stimulating factor for acute ischemic stroke: a randomized controlled trial. CMAJ, 2006. 174(7): p. 927-33.
35. Yata, K., et al., Granulocyte-colony stimulating factor inhibits apoptotic neuron loss after neonatal hypoxia-ischemia in rats. Brain Res, 2007. 1145: p. 227-38.
36. Komine-Kobayashi, M., et al., Neuroprotective effect of recombinant human granulocyte colony-stimulating factor in transient focal ischemia of mice. J Cereb Blood Flow Metab, 2006. 26(3): p. 402-13.
37. Shomali, M. E., The use of anti-aging hormones. Melatonin, growth hormone, testosterone, and dehydroepiandrosterone: consumer enthusiasm for unproven therapies. Md Med J, 1997. 46(4): p. 181-6.
38. Sirohi, B., et al., Use of physiological doses of human growth hormone in haematological patients receiving intensive chemotherapy promotes haematopoietic recovery: a double-blind randomized, placebo-controlled study. Bone Marrow Transplant, 2007. 39(2): p. 115-20.
39. Deng, Z. R., et al., [Dynamic changes of plasma VEGF, SDF-1 and peripheral CD34+ cells in patients with acute myocardial infarction]. Nan Fang Yi Ke Da Xue Xue Bao, 2006. 26(11): p. 1637-40.
40. Grundmann, F., et al., Differential increase of CD34, KDR/CD34, CD133/CD34 and CD117/CD34 positive cells in peripheral blood of patients with acute myocardial infarction. Clin Res Cardiol, 2007. 96(9): p. 621-7.
41. Kissel, C. K., et al., Selective functional exhaustion of hematopoietic progenitor cells in the bone marrow of patients with postinfarction heart failure. J Am Coll Cardiol, 2007. 49(24): p. 2341-9.
42. Abbott, J. D., et al., Stromal cell-derived factor-1alpha plays a critical role in stem cell recruitment to the heart after myocardial infarction but is not sufficient to induce homing in the absence of injury. Circulation, 2004. 110(21): p. 3300-5.
43. Nakamura, T., et al., Significance and therapeutic potential of endothelial progenitor cell transplantation in a cirrhotic liver rat model. Gastroenterology, 2007. 133(1): p. 91-107 e1.
44. Togel, F., et al., Renal SDF-1 signals mobilization and homing of CXCR4-positive cells to the kidney after ischemic injury. Kidney Int, 2005. 67(5): p. 1772-84.
45. Lemoli, R. M., et al., Mobilization of bone marrow-derived hematopoietic and endothelial stem cells after orthotopic liver transplantation and liver resection. Stem Cells, 2006. 24(12): p. 2817-25.

1. A method of augmenting stem cell numbers in circulation comprising administering to a mammal a composition comprising of one or more ingredients selected from the group consisting of: a) Ashwagandha; b) Milk Thistle; c) Vitamin D3; d) Fucoidan; and e) Garcinia Indica.

2. The method of claim 1, wherein said composition comprises approximately 50-300 mg Ashwagandha, 500-2000 IU Vitamin D3, 100-1000 mg Fucoidan, and 50-500 mg of Garcinia Indica.

3. The method of claim 1, wherein said composition comprises approximately 100 mg Ashwagandha, 1000 IU Vitamin D3, 500 mg Fucoidan, and 250 mg of Garcinia Indica.

4. The method of claim 1, wherein said composition is administered between 1 time per day, to 10 times per day.

5. The method of claim 1, wherein said composition is administered approximately 2 times per day.

6. The method of claim 1, wherein said composition is administered to a patient with low circulating endothelial progenitor cells and dose of said composition is administered according to desire to increase circulating endothelial progenitor cells.

7. The method of claim 6, wherein said endothelial progenitor cells are cells capable of forming endothelial colonies in vitro.

8. The method of claim 6, wherein said endothelial progenitor cells express a marker selected from the group consisting of: a) CD34; b) CD133; c) KDR-1; d) stem cell antigen; e) CD146; f) CD31; g) Tie-2; h) CD144; and i) VEGFR3.

9. The method of claim 6, wherein said endothelial progenitor cells are capable of stimulating neoangiogenesis.

10. The method of claim 6, wherein said endothelial progenitor cells are capable of stimulating neoangiogenesis in an immune deficient animal.

11. The method of claim 1, wherein said composition further comprises an antioxidant.

12. The method of claim 11, wherein said antioxidant is selected from the group consisting of: a) 1-carnosine; b) a carotenoid; c) co-enzyme Q10; d) green tea or extract thereof; e) vitamin A; f) vitamin B; g) vitamin c; h) vitamin E; i) selenium; j) resveratrol; and k) lipoic acid.

13. The method of claim 1, wherein said composition is delivered in a route selected from the group consisting of: oral, liquid, suppository, and transdermal form.

14. The method of claim 1, wherein ingredient of said composition is concentrated using an extraction procedure.

15. The method of claim 14, wherein said ingredient is capable of increasing the number of stem cells in circulation.

16. The method of claim 1, wherein said stem cell is selected from the group consisting of: a) hematopoietic stem cells; b) circulating endothelial progenitor cells; c) circulating mesenchymal stem cells; d) circulating very small embryonic like cells; and e) circulating aldehyde dehydrogenase bright cells.

17. The method of claim 16, wherein said hematopoietic stem cells are capable of stimulating production of new blood cells in vitro.

18. The method of claim 16, wherein said hematopoietic stem cells are committed progenitor cells.

19. The method of claim 16, wherein said hematopoietic stem cells are myeloid progenitor cell.

20. The method of claim 16, wherein said hematopoietic stem cells are lymphoid progenitor cells.

21. The method of claim 16, wherein said hematopoietic stem cells are cells expressing a marker selected from the group consisting of: a) CD34, b) CD133, and c) stem cell antigen.

22. A method of increasing efficacy of a mobilizing agent comprising administration of said mobilizing agent together with a composition comprising one or more ingredients selected from the group consisting of: a) Ashwagandha; b) Milk Thistle; c) Vitamin D3; d) Fucoidan; and e) Garcinia Indica.

23. A method of increasing efficacy of an allogeneic or an autologous stem cell therapy, said method comprising administration of a mobilizing agent together with a composition comprising one or more ingredients selected from the group consisting of: a) Ashwagandha; b) Milk Thistle; c) Vitamin D3; d) Fucoidan; and e) Garcinia Indica.
